(19) United States
(12) Patent Application Publication
(10) Pub. No.: US 2010/0330168 A1
(43) Pub. Date: Dec. 30, 2010

(54) PHARMACEUTICAL COMPOSITION BASED ON MICRONIZED PROGESTERONE AND USES THEREOF

(75) Inventors: Jennifer Gicquel, Orgeval (FR); Didier Terracol, Verrieres Le Buisson (FR); Roland Samoyeau, Antony (FR)

Correspondence Address: DICKSTEIN SHAPIRO LLP, 1633 Broadway, NEW YORK, NY, 10019, US

(73) Assignee: EFFIK, Bievres (FR)

(21) Appl. No.: 12/824,489

(22) Filed: Jun. 28, 2010

(30) Foreign Application Priority Data
Jun. 29, 2009 (FR) 0954420

(51) Int. Cl. A61K 31/575; A61K 31/57; A61K 9/48; A61K 31/566; A61K 31/565; A61K 31/567; A61P 5/30

(52) U.S. Cl. 424/451; 514/177; 514/170

(57) ABSTRACT

Novel pharmaceutical compositions contain micronized progesterone and at least one oleic safflower oil referred to as safflower oil type II, and medicines comprising said compositions are useful in treating progesterone insufficiency in women.

TABLE OF CONTENTS

| Background of the Invention | | Page |
|---|---|---|
| 1. | Field of the Invention | 1 |
| 2. | Description of Related Art | 2 |

| | |
|---|---|
| Summary of the Invention | 3 |
| Brief Description of the Drawing | 3 |
| Detailed Description | 4 |

| | |
|---|---|
| Example 1 | 6 |
| Example 2 | 8 |
| Example 3 | 9 |
| Example 4 | 10 |
| Example 5 | 11 |

| | |
|---|---|
| Claims | 12 |
| Abstract of the Disclosure | 14 |
| Drawing | 15 |

### BACKGROUND OF THE INVENTION

1. Field of the Invention

The present subject matter relates to pharmaceutical compositions containing micronized progesterone and an oleic safflower oil, referred to herein as safflower oil type II. It also relates to medicines and products comprising the said pharmaceutical compositions.

The present subject matter also relates to uses of the pharmaceutical compositions.

2. Description of Related Art

Progesterone is a hormone which is synthesized in human ovaries, more specifically in the cells of the corpus luteum, in varying plasma concentrations during the luteal phase of the menstrual cycle. To a lesser extent, progesterone is produced by the adrenal glands and the placenta during the second trimester of pregnancy.

Progesterone is secreted in greater amounts from the 14th day of the menstrual cycle by the cells of the granulosa of the corpus luteum. It enables the maintenance and densification of the uterine mucosa until implantation of the egg in the uterus, the development of endometrial vascularisation, and the formation of uterine glands responsible for the serrated aspect of the uterine wall.

During pregnancy, progesterone prepares the mammary glands for lactation and acts as a natural relaxant so as to prevent uterine contractions. If there is no fertilization, the progesterone level returns to normal.

Progesterone secretion insufficiency, known as luteal insufficiency, may be responsible for many pathological conditions, in particular successive miscarriages, menstrual cycle disturbances, and premenstrual syndrome.

Dietary or nutritional supplementation with progesterone makes it possible to treat luteal insufficiencies, but oral administration of progesterone has suffered from certain limitations since progesterone is weakly absorbed by the intestine and, in addition, has a relatively short plasma half-life. In fact, vaginal, rectal, and intramuscular routes of administration appear to be more suitable for maintaining, for several consecutive hours, a progesterone concentration at the physiological level of the luteal phase.

A formulation containing micronized progesterone in an oily suspension, thereby making it possible to increase the bioavailability of the progesterone when administered orally, is disclosed in published French Patent Application 76 36007 (corresponding to published British Patent Specification 1 595 185 and U.S. Pat. No. 4,196,188). This formulation is marketed in France under the trade mark UTROGESTAN®.

However, the oil used in UTROGESTRAN® formulation is peanut oil, which is known to possess allergenic properties and to cause considerable hypersensitivity reactions. U.S. Pat. No. 4,900,734 describes compositions containing safflower oil type I, sunflower oil, corn oil, linseed oil, or mixtures of these oils, micronized progesterone and an oestradiol, for treating menopausal symptoms.

Published Patent Application WO 03/041720 discloses replacing peanut oil with other oils such as sunflower oil, olive oil, sesame oil, rapeseed oil or almond oil, which make it possible to suppress the risks of allergic reactions while at the same time conserving all the physico-chemical characteristics of the UTROGESTAN® formulation. However, these formulations still contain a certain proportion of soybean lecithin, which is also known to possess considerable allergenic properties. This lipid is in fact capable of inducing an immune response that carries with it the risk of hypersensitive reactions (anaphylactic shock, urticaria). This risk has led public health authorities to define soybean lecithin as an excipient “with a recognized pharmacological effect” in the Summary of Product Characteristics for Utrogestan®.

There remains therefore a need for a micronized progesterone formulation which does not have drawbacks in terms of allergenicity.

### SUMMARY OF THE INVENTION

The applicants have discovered that, by using safflower oil type II, a composition can be formulated which is free of soybean lecithin while at the same time conserving the dispersion qualities of the micronized progesterone in medicines.

The present subject matter therefore includes a pharmaceutical composition comprising micronized progesterone in safflower oil type II.

The present subject matter also includes the use of micronized progesterone in safflower oil type II for the treatment of physiological conditions related to progesterone secretion insufficiency.

Other subjects matter will become apparent from the following description and examples.

### BRIEF DESCRIPTION OF THE DRAWING

FIG. 1 shows the linearity between the measured area of the peak and the amount of progesterone dissolved in an oily medium. The limit of detection (LOD) of progesterone is 0.38 micrograms/ml and the limit of quantification (LOQ) of progesterone is 1.16 micrograms/ml.

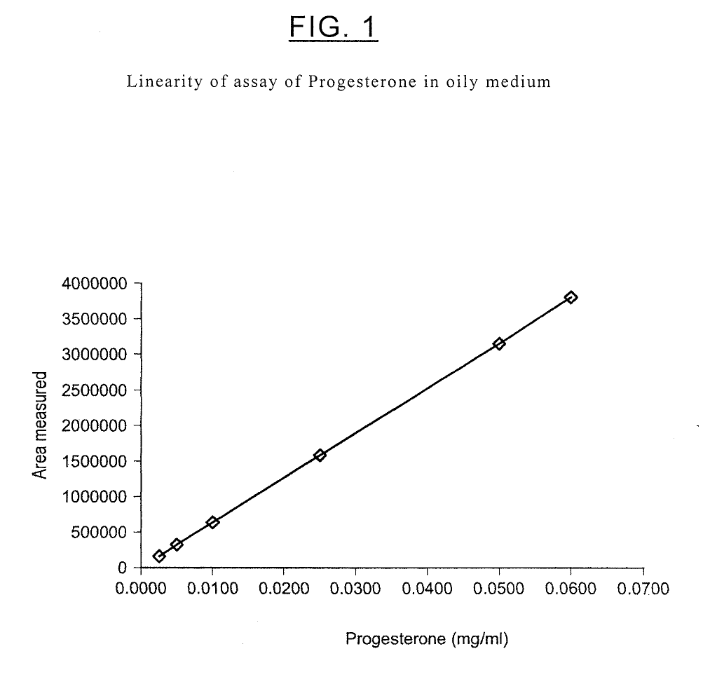

The value of the peak in FIG. 1 has not unity. Indeed, it displays intrinsic values calculated by the chromatograph calculator, which analyzes the response of this system according to the amount of progesterone. The plot in FIG. 1 represents the synthesis of 6 chromatographs of progesterone.

The linearity of the plot in FIG. 1 demonstrates that the method of dosage of progesterone is reliable independently of the concentration (between 0 and 0.006 mg/ml). The LOD and the LOQ do not appear in FIG. 1, but rather correspond to quality parameters of the chromatograph system (validation parameters of the method).

### DETAILED DESCRIPTION

The inventors have discovered that, among the available oils, safflower oil type II does not pose a risk of allergic reaction and offers the best nutritional profile. The applicants have also found that safflower oil type II provides a considerable technical benefit insofar as it makes it possible to suppress the potentially allergenic soybean lecithin in the composition, without impairing the quality of the suspension of the micronized progesterone in the oil during the preparation of the composition.

The choice of safflower oil type II makes it possible to better control the rate of sedimentation of progesterone in oil. In the present pharmaceutical compositions, micronized progesterone is thus present in suspension in the safflower oil type II.

Safflower oil type II is extracted from safflower seeds, safflower being a plant that originates from Egypt. It is also found in the wild in China, India, Japan, Australia and Iran and throughout America. Safflower oil type II is extremely advantageous from a nutritional point of view, since it is rich in unsaturated essential fatty acids, including linoleic acid and oleic acid. Furthermore, it contains very low levels of saturated fats (saturated fatty acids).

The average fatty acid composition of safflower oil type II is approximately from 65% to 85% of oleic acid and from 5% to 25% of linoleic acid. This oil is thus an important source of monounsaturated fatty acids, particularly oleic acid which is necessary for re-establishing the omega-6/omega-3 balance because of its neutrality with respect to essential fatty acid metabolism enzymes.

In the context of the present subject matter, the safflower oil type II used may be refined or unrefined. A refined oil is an oil which is obtained starting from the crude oil which has undergone a set of refining operations. The refined oil is a purified oil which has a very low content of impurities and is free—in particular—of potentially allergenic proteins such as gluten.

Suitable safflower oil type II is available from the Olisud Company, Aix en Provence, France (refined oleic safflower oil CAS 8001-23-8, December 2009).

In the context of the present subject matter, the term “micronized progesterone” means a progesterone compound in particulate form in which at least 99% of the particles have a particle size of less than 60 μm, said particle size being measured by using a Malvern laser particle sizer according to a known method (Delgrove R., and J. M. Hochart, AVH Association, 8th symposium Reims, March 2001).

The progesterone/oil weight ratio is desirably between about 0.15/1 and about 3/1, preferably between about 0.25/1 and about 2/1, more preferably between about 0.40/1 and 1/1, and, even more preferentially, about 0.67/1.

Pharmaceutical compositions according to the present subject matter can be prepared by gradually adding the micronized progesterone to the safflower oil type II, with stirring at approximately 1000 rpm, and then maintaining the stirring at the same speed for approximately one hour in order to obtain a homogeneous suspension.

The pharmaceutical compositions according to the present subject matter can also comprise an oestrogen or an ester-type derivative thereof, preferably selected from the group consisting of 17β-oestradiol, oestrone, 17α-ethinyloestradiol, oestradiol valerate or phyto-oestrogens, and even more preferentially 17β-oestradiol.

The pharmaceutical compositions according to the present subject matter contain a suspending agent such as fumed silica as is available under the trademark AEROSIL® (Aerosil R972 Pharma, France) desirably at an amount of from about 0.1% to about 1% by weight of the composition, preferably from about 0.4% to about 0.6%, and even more preferably at a percentage of about 0.5% by weight of the composition.

The pharmaceutical compositions according to the present subject matter can be in the form of a soft capsule comprising binders, disintegrating agents, diluents and/or lubricants.

According to one advantageous process for manufacturing the pharmaceutical compositions according to the subject matter, the capsule comprises gelatine or a similar component.

When the present pharmaceutical composition is integrated into a pharmaceutical specialty product, each dosage unit advantageously comprises between about 2 mg and about 600 mg of micronized progesterone, and preferably between about 100 mg and about 400 mg.

The pharmaceutical compositions according to the present subject matter can be administered orally or vaginally, according to the therapeutic indications.

Vaginal administration also represents an alternative to oral administration in the event of adverse effects due to progesterone (drowsiness after oral absorption) or if oral administration is contraindicated (hepatopathy).

The micronized progesterone in safflower oil type II is thus useful in the treatment of physiological conditions related to progesterone secretion insufficiency.

The present subject matter also encompasses the use of micronized progesterone in safflower oil type II for the preparation of medicines for the treatment of physiological conditions related to progesterone secretion insufficiency.

Examples of such physiological conditions include luteal insufficiency, menstrual irregularity, premenstrual syndrome, mastodynia, benign mastopathies, the premenopause period, sterility caused by luteal insufficiency, menopausal disturbances, local contraception, the prevention of recurrent abortions in the event of luteal insufficiency, threatened premature delivery, osteoporosis, hyperplasia and cancer of the endometrium, and epilepsy.

The present subject matter also relates to the use of micronized progesterone and safflower oil type II, in combination with an oestrogen, for the preparation of medicines for the treatment of physiological conditions related to progesterone secretion insufficiency. The oestrogen or an ester-type derivative thereof is preferably selected from the group constituted of 17β-oestradiol, oestrone, 17α-ethinyloestradiol, oestradiol valerate or phyto-oestrogens, and even more preferentially 17β-oestradiol.

The following non-limiting examples illustrate the present subject matter.

### Example 1

### Measurement of Sedimentation by Centrifugation of Progesterone in the Composition According to the Present Subject Matter

Various oil/micronized progesterone mixtures were prepared by introducing x milliliters of test oil into a 600 milliliter beaker, with stirring at 1000 rpm with a butterfly blade, while gradually adding the micronized progesterone. The mixtures were then stirred for one hour at 1000 rpm with a butterfly blade. The oils used were obtained from Olisud Company (refined peanut oil batch L805160, refined evening primrose oil batch L710020, refined grape seed oil batch L804231, refined oleic safflower oil referred to as refined safflower oil type II, batch L709271). The micronized progesterone no. 429 of the European Pharmacopoeia, current edition, including supplements, was obtained from Zhejiang Shenzhou Pharmaceutical Co., Ltd., 14 Chuancheng Nan Road, Xianju 317300, Zejiang, China, batch Prob-080304.

The sedimentation test is then carried out by centrifugation of the mixtures at 4000 rpm for periods of 10, 15 and 20 minutes. The supernatant was removed by drawing it up following centrifugation and the average volume was then determined, measured, and weighed at various times after centrifugation. The weight is converted to volume by dividing the latter by the density of the oil under consideration, and the average regression slope is calculated in milliliters per minute.

TABLE 1
Measurement of progesterone sedimentation according
to the oils used

| | Safflower | Sun- | | | | Safflower |
|---|---|---|---|---|---|---|
| Oil | II | flower | Olive | Peanut | Rapeseed | I |
| Test 1: | | | | | | |
| volumes at | | | | | | |
| −10 min | 1.21 | 1.62 | 1.45 | 1.65 | 1.72 | 1.83 |
| (ml) | | | | | | |
| −15 min | 2.22 | 2.52 | 2.73 | 2.97 | 3.34 | 3.42 |
| (ml) | | | | | | |
| −20 min | 3.29 | 4.00 | 4.33 | 4.51 | 5.09 | 5.12 |
| (ml) | | | | | | |
| Slope | 0.209 | 0.238 | 0.288 | 0.286 | 0.337 | 0.329 |
| (ml/min) | | | | | | |
| Test 1: | | | | | | |
| volumes at | | | | | | |
| −10 min | 1.13 | 1.58 | 1.40 | 1.62 | 1.70 | 1.75 |
| (ml) | | | | | | |
| −15 min | 2.19 | 2.49 | 2.70 | 2.86 | 3.35 | 3.33 |
| (ml) | | | | | | |
| −20 min | 3.39 | 4.13 | 4.25 | 4.39 | 4.69 | 5.03 |
| (ml) | | | | | | |
| Slope | 0.226 | 0.254 | 0.285 | 0.277 | 0.300 | 0.328 |
| (ml/min) | | | | | | |
| Average | 3.34 | 4.07 | 4.29 | 4.45 | 4.89 | 5.08 |
| volume at | | | | | | |
| 20 min | | | | | | |
| (ml) | | | | | | |
| Average | 0.218 | 0.246 | 0.286 | 0.281 | 0.318 | 0.329 |
| slope | | | | | | |
| (ml/min) | | | | | | |

Table 1 clearly shows that the smallest amount of progesterone sedimentation is obtained with the safflower oil type II, by comparison with peanut oil, sunflower oil, rapeseed oil, olive oil and safflower oil type I.

### Example 2

### Measurement of the Viscosity of the Composition According to the Invention

Various oil/micronized progesterone mixtures were prepared by introducing x milliliters of test oil into a 600 milliliter beaker, with stirring at 1000 rpm with the butterfly blade, while at the same time gradually adding the micronized progesterone. The mixtures are then stirred for one hour at 1000 rpm with a butterfly blade. The oils used come from Olisud Company (refined peanut oil batch L805160, refined evening primrose oil batch L710020, refined grape seed oil batch L804231, refined oleic safflower oil referred to as refined safflower oil type II, batch L709271). The micronized progesterone comes from Shenzhou, Zejiang, China, batch Prob-080304.

For these measurements, the volume of the sample tested is 30 grams±0.5 g at a temperature of 22° C. for each mixture; the viscosity is measured after mixing and after standing for approximately two hours.

TABLE 2
Measurement of the viscosity of the oils used (mPa · s)

| | | | | | | Safflower |
|---|---|---|---|---|---|---|
| Oil | Rapeseed | Safflower I | Sunflower | Olive | Peanut | II |
| T0 | 5590 | 5740 | 6210 | 6210 | 6850 | 6550 |
| T2h | 5110 | 5780 | 6620 | 6750 | 7050 | 7350 |

Table 2 thus shows that the viscosities are substantially equivalent according to the oils used, the viscosity of the safflower oil type II being very close to that of the peanut oil.

### Example 3

### Comparison of the Oils According to the Invention with a Formulation Similar to that of Utrogestan®

The physico-chemical parameters of the safflower oil type II were tested in comparison with a formulation containing peanut oil and 0.4% of soybean lecithin, according to the protocol previously described.

The results are shown in Table 3 below.

TABLE 3
Comparison of the oils taking into account
the presence of soybean lecithin

| | | Sedimentation (average |
|---|---|---|
| | Viscosity at T 2 h | volume in mL after 20 |
| | (mPa · s) | minutes) |
| Peanut oil + 0.4% | 6610 | 3.61 |
| soybean lecithin | | |
| Safflower oil type II | 7350 | 3.34 |

It is thus seen that the change of the oil in the micronized progesterone formulations does not affect the physico-chemical properties of the compositions of the present subject matter.

### Example 4

### Assay of Solubilized Progesterone in the Composition According to the Invention

Various oil/micronized progesterone mixtures were prepared by introducing x milliliters of test oil into a 600 milliliter beaker, with stirring at 1000 rpm with a butterfly blade, while gradually adding the micronized progesterone. The mixtures were then stirred for one hour at 1000 rpm with a butterfly blade. The oils used were from Olisud Company (refined peanut oil batch L805160, refined evening primrose oil batch L710020, refined grape seed oil batch L804231, refined oleic safflower oil referred to as refined safflower oil type II, batch L709271). The micronized progesterone comes from Shenzhou, Zejiang, China, batch Prob-080304.

The solubilization test is carried out by centrifugation of the mixtures at 4000 rpm for periods of 10, 15 and 20 minutes. The solubilized progesterone is then assayed on a test sample of the total supernatant collected, after centrifugation, from the samples of Example 1.

TABLE 4
Assay of solubilized progesterone

| | Content of solubilised |
|---|---|
| | progesterone |
| Formulation | in oil as % m/m |
| Refined peanut oil + soybean lecithin control | 1.86 |
| Refined peanut oil | 1.84 |
| Refined safflower oil type II | 1.90 |
| Refined evening primrose oil | 2.15 |
| Refined grape seed oil | 2.14 |
| Sunflower oil | 2.13 |

Table 4 shows that the mixture containing safflower oil type II has a content of solubilized progesterone similar to that of the mixtures comprising refined peanut oil with or without soybean lecithin.

### Example 5

### Pharmaceutical Compositions According to the Invention

| | Viscosity at T = 0 | Sedimentation |
|---|---|---|
| Composition formulations | (mPa · s) | (ml/mm) |
| Progesterone 100 mg | 6710 | 0.255 |
| Oleic safflower oil type II 150 mg | | |
| Progesterone 100 mg | 5980 | 0.257 |
| Oleic safflower oil type II 149.4 mg | | |
| Aerosil 0.625 mg | | |
| Control 1 | 7110 | 0.285 |
| Progesterone 100 mg | | |
| Peanut oil 150 mg | | |
| Control 2 | 6650 | 0.257 |
| Progesterone 100 mg | | |
| Peanut oil 149 mg | | |
| Soybean lecithin 1 mg | | |

1. A pharmaceutical composition comprising micronized progesterone and safflower oil type II.

2. A composition according to claim 1 wherein the progesterone/oil weight ratio is between about 0.15/1 and about 3/1.

3. A composition according to claim 1 wherein the progesterone/oil weight ratio is between about 0.25/1 and about 2/1.

4. A composition according to claim 1 wherein the progesterone/oil weight ratio is between about 0.40/1 and about 1/1.

5. A composition according to claim 1 wherein the progesterone/oil weight ratio is about 0.67/1.

6. A composition according to claim 1, wherein the progesterone is in suspension in the oil.

7. A composition according to claim 1, further comprising an oestrogen or an ester-type derivative thereof.

8. A composition according to claim 7 wherein the derivative is selected from the group consisting of 17β-oestradiol, oestrone, 17α-ethinyloestradiol, oestradiol valerate or phyto-oestrogens.

9. A composition according to claim 8 wherein the derivative is 17β-oestradiol.

10. A composition according to claim 1, further comprising a suspending agent.

11. A composition according to claim 10, wherein the suspending agent is fumed silica.

12. A composition according to claim 1, wherein said composition is in the form of a soft capsule.

13. A pharmaceutical product comprising a composition according to claim 1, wherein said product is in the form of a dosage unit in the range of between about 2 mg and about 600 mg of micronized progesterone.

14. A product according to claim 13 wherein said dosage unit is between about 100 mg and about 400 mg.

15. A method of treating a physiological condition related to progesterone secretion insufficiency, comprising administering an effective amount of a medicine comprising micronized progesterone in safflower oil type II.

16. A method according to claim 15 wherein the medicine additionally contains an oestrogen or ester-type derivative thereof.

17. A method according to claim 15 wherein the oestrogen or ester-type derivative thereof is selected from the group consisting of 17β-oestradiol, oestrone, 17α-ethinyloestradiol, oestradiol valerate or phyto-oestrogens.

18. A method according to claim 17 wherein the oestrogen or ester-type derivative thereof is 17β-oestradiol.

19. A method according to claim 14 or 20 wherein the medicine additionally contains a suspending agent.

20. A method according to claim 19 wherein the suspending agent is fumed silica.
